(19) United States
(12) Patent Application Publication
(10) Pub. No.: US 2010/0086607 A1
(43) Pub. Date: Apr. 8, 2010

(54) Self-Assembled Biodegradable Nanoparticles for Medical and Biological Applications

(75) Inventor: Esmaiel Jabbari, Columbia, SC (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/575,876

(22) Filed: Oct. 8, 2009

Related U.S. Application Data
(60) Provisional application No. 61/195,627, filed on Oct. 8, 2008.

(51) Int. Cl. A61K 9/14; C08F 8/30; C08L 1/00; A61K 47/48

(52) U.S. Cl. 424/489; 525/375; 514/2; 977/773; 977/906

(57) ABSTRACT

A method for forming a biodegradable composition that self-assembles into nanoparticles is provided. The method includes reacting N,N′-Disuccinimidyl carbonate with hydroxyl end-groups of poly(lactide-co-fumarate) to form a composition comprising succinimide-terminated poly(lactide-co-fumarate).

### CROSS REFERENCE TO RELATED APPLICATION

The present application claims is based on and claims priority to U.S. Provisional Application Ser. No. 61/195,627, filed Oct. 8, 2008, which is incorporated by reference herein in its entirety.

### GOVERNMENT SUPPORT CLAUSE

The present invention was developed with funding from the National Institutes of Health under award P20 RR-016461 from the National Center for Research Resources and by the National Science Foundation/EPSCoR under Grant No. 2001 RII-EPS-0132573. Therefore, the government retains certain rights in this invention.

### BACKGROUND

Biodegradable nanoparticles (NPs) are being explored for a wide range of applications in medicine including in-vivo imaging and diagnosis, as biosensors for molecular recognition, and as carriers for drug delivery and gene therapy. In drug delivery, NP carriers substantially improve drug stability and allow sustained release of therapeutic agents which increase drug bioavailability and reduce the dosing frequency. For example, NPs have been used in targeted tumor delivery to circumvent the short half-life and limited solubility of chemotherapeutic agents and improve selectivity. Biodegradable NPs, surface-modified with hydrophilic polymers, can evade the mononuclear phagocytes system (MPS), overcome resistance at the tumor level (EPR effect), and can be conjugated with ligands with high specificity to tumor cells to localize the NPs and increase the effective concentration of the antitumor drug in the tumor microenvironment. In vaccination, the use of NPs for sustained delivery of the antigens can reduce dosing frequency and side effects. Their large surface area and small size make biodegradable NPs also attractive as a carrier for stabilization and sustained delivery of proteins in tissue engineering applications. For example, physiosorption of recombinant human bone morphogenetic protein-2 (rhBMP-2) on poly(lactide-co-glycolide) (PLGA) NPs has resulted in enhanced bone formation in-vivo in rat calvarial defect model.

Poly(lactide-co-glycolide) (PLGA) polymers have been used to produce NPs by emulsion-solvent extraction for delivery of therapeutic agents. The drug release characteristics (short- or long-term) from PLGA NPs can be tailored to a particular application by varying the ratio of lactide to glycolide in the copolymer. However, drug loading in PLGA NPs is generally low and protein drugs have significant solubility in organic solvents used in emulsion-solvent extraction technique. Consequently, a large fraction of the protein is denatured in the emulsification process which reduces the drug therapeutic efficacy. Furthermore, non-degradable surfactants are used to stabilize the NPs suspension which affects the physical and biological properties of the NPs. PLGA polymers grafted with 1-5 wt % poly(ethylene glycol) units are used to produce NPs without the use of non-degradable surfactants like polyvinyl alcohol. However, the use of organic solvents limits the NPs to encapsulation of non-protein drugs.

Linear and star Poly(L-lactide)-b-poly(ethylene oxide) copolymers have been used to produce degradable NPs. The PLA-PEG and PLGA-PEG NPs can be loaded with a variety of bioactive agents and the ethylene oxide (EO) blocks in the copolymer act as a surfactant to stabilize the NPs. However, the EO units impart hydrophilicity to the copolymer, which limits the duration of release of therapeutic agents from the NPs to short-term (1-2 days). Poly(vinyl alcohol)-graft-PLGA NPs have been used for local delivery of Paclitaxel in restenosis treatment but an initial burst release dominated the early release profile followed by a slow continuous release of a small fraction of the drug. Amino cyclodextrin conjugated PLGA polymer has been used to produce biodegradable NPs as a carrier for delivery of proteins. The entrapment of bovine serum albumin (BSA) in the NPs was as high 50% and the protein release was in three phases: a burst effect in the first day, followed by a plateau for one week, and a sustained release phase for two weeks.

There remains a need to develop biocompatible NP carriers that allow sustained release of small bioactive molecules and protein drugs while degrading concurrent with the release of the active agent.

### SUMMARY

Objects and advantages of the invention will be set forth in part in the following description, or may be obvious from the description, or may be learned through the practice of the invention.

In accordance with certain embodiments of the present disclosure, a method for forming a biodegradable composition that self-assembles into nanoparticles is provided. The method includes reacting N,N′-Disuccinimidyl carbonate with hydroxyl end-groups of poly(lactide-co-fumarate) to form a composition comprising succinimide-terminated poly(lactide-co-fumarate).

In certain aspects of the present disclosure, the succinimide-terminated poly(lactide-co-fumarate) can be combined with poly(lactide-co-ethylene oxide-co-fumarate) in a solvent solution and dialysis can be performed of the solution so that the composition self-assembles into nanoparticles.

In another embodiment of the present disclosure, a method for forming a biodegradable composition that self-assembles into nanoparticles is provided in which N,N′-Disuccinimidyl carbonate is reacted with hydroxyl end-groups of poly(lactide-co-glycolide-co-fumarate) to form a composition comprising succinimide-terminated poly(lactide-co-glycolide-co-fumarate).

In certain aspects of the present disclosure, the succinimide-terminated poly(lactide-co-glycolide-co-fumarate) can be combined with poly(lactide-co-ethylene oxide-co-fumarate) in a solvent solution and dialysis can performed of the solution so that the composition self-assembles into nanoparticles.

In yet another embodiment of the present disclosure, a biodegradable composition that self-assembles into nanoparticles is provided, the composition comprising succinimide-terminated poly(lactide-co-fumarate).

In still another embodiment of the present disclosure, a biodegradable composition that self-assembles into nanoparticles is provided, the composition comprising succinimide-terminated poly(lactide-co-glycolide-co-fumarate).

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates a scheme for the synthesis of succinimide-terminated PLAF-NHS macromer by reacting the hydroxyl end-group of the PLAF with the carbonate group of DSC, in accordance with certain aspects of the present disclosure.

FIG. 2 illustrates ¹H-NMR spectra of PLAF and PLAF-NHS, in accordance with certain aspects of the present disclosure.

FIG. 3 illustrates a SEM image of PLAF-NHS NPs (90% PLAF-NHS and 10% PLEOF) showing the spherical shape and smooth surface morphology of the NPs, in accordance with certain aspects of the present disclosure. The image in shows the size distribution of the NPs. The scale bars in the image and insert are 1 μm are 500 nm, respectively.

FIG. 4 illustrates the effect of PLA fraction in PLEOF macromer on the average size of PLAF/PLEOF NPs (90% PLAF and 10% PLEOF), in accordance with certain aspects of the present disclosure.

FIG. 5 illustrates a comparison of the size distribution of PLAF and PLGF NPs (90% PLAF or PLGF and 10% PLEOF), in accordance with certain aspects of the present disclosure. The average size of the PLAF and PLGF NPs was 325 and 250 nm, respectively, with breadth of distribution (one standard deviation from the mean) of 135 and 115 nm.

FIG. 6 illustrates the effect of incubation time (0, 1, 2, and 7 days) on size distribution of PLAF (a) and PLGF (b) NPs (90% PLAF or PLGF and 10% PLEOF), in accordance with certain aspects of the present disclosure.

FIG. 7 illustrates the effect of incubation time on the average size of the PLAF and PLGF NPs (90% PLAF or PLGF and 10% PLEOF), in accordance with certain aspects of the present disclosure.

FIG. 8 illustrates a schematic diagram of the core-shell model for the structure of PLGF/PLEOF blend NPs, in accordance with certain aspects of the present disclosure.

FIG. 9 illustrates the effect of incubation time on mass loss of the PLAF and PLGF NPs (90% PLAF or PLGF and 10% PLEOF), in accordance with certain aspects of the present disclosure. The effect of incubation time on water uptake of PLAF, PLGF, and PLEOF disks is shown.

FIG. 10 illustrates release characteristics of PAN encapsulated in PLAF and PLGF NPs (90% PLAF or PLGF and 10% PLEOF), in accordance with certain aspects of the present disclosure.

FIG. 11 illustrates release characteristics of BSA conjugated to PLAF-NHS and PLGF-NHS NPs (90% PLAF-NHS or PLGF-NHS and 10% PLEOF), in accordance with certain aspects of the present disclosure.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

Generally, the present disclosure provides an improved method for fabrication of biodegradable and shape-specific polymeric scaffolds. Such scaffolds can include well-defined pore geometry, functionalized with covalently attached bioactive peptides, for applications in tissue regeneration.

Novel poly(lactide-co-fumarate) (PLAF), poly(lactide-co-glycolide-co-fumarate) (PLGF), and poly(lactide-co-ethylene oxide-co-fumarate) (PLEOF) unsaturated macromers that self-assemble to form biodegradable NPs are described herein. The lactide and glycolide units are FDA approved for certain clinical applications, ethylene oxide units are excreted through the kidneys, and fumaric acid units occur naturally in the Kreb's cycle. The degradation characteristics of the NPs can be adjusted by the ratio of lactide to glycolide in the PLGF macromer. The degree of hydrophilicity, hence their circulation half-life, can be controlled by the molecular weight and fraction of poly (ethylene glycol) (PEG) in the macromer. NPs ranging 50-500 nm in size can be produced by varying the ratio of PLEOF to PLAF or PLGF in the blend. In the process of NPs formation, biodegradable PLEOF macromer is used as a surfactant to stabilize the NPs. The unsaturated fumarate groups can be used to covalently attach ligands to PLGF/PLEOF NPs for targeted tumor delivery or biomolecular recognition. The chain-ends of the macromer can be functionalized with succinimide (NHS) groups for conjugation of proteins to NPs, as opposed to encapsulating. N,N′-Disuccinimidyl carbonate (DSC) is reacted with hydroxyl end-groups of the PLAF and PLGF macromers to produce succinimide-terminated PLAF-NHS and PLGF-NHS macromers.

The present disclosure compares the release characteristics of model molecules encapsulated in PLAF or PLGF NPs with those conjugated to PLAF-NHS or PLGF-NHS NPs. 1-(2-pyridylazo)-2-naphthol (PAN) was used as a model small molecule for encapsulation in PLAF and PLGF NPs and bovine serum albumin (BSA) was used as a model protein for conjugating to PLAF-NHS and PLGF-NHS. NPs were produced by dialysis of the macromers in dimethylsulfoxide (DMSO)/N,N-dimethylformamide (DMF) against water with amphiphilic PLEOF macromer used as surfactant to stabilize the NPs. PAN was encapsulated in PLAF or PLGF NPs in the process of dialysis. BSA was conjugated to PLAF-NHS or PLGF-NHS NPs after self-assembly of the NPs by dialysis. The release profile of the encapsulated PAN from PLAF and PLGF NPs was non-linear and consisted of a burst release in the first 24 h followed by a period of sustained release. The release profile for BSA conjugated to PLAF-NHS and PLGF-NHS was linear up to complete degradation of the NPs. PLGF and PLAF NPs degraded in 15 and 28 days, respectively, while PLGF-NHS and PLAF-NHS NPs degraded in 25 and 38 days, which demonstrated that the release was dominated by erosion of the matrix.

The following examples are meant to illustrate the disclosure described herein and are not intended to limit the scope of this disclosure.

### EXAMPLES

Lactide-co-glycolide based functionalized nanoparticles (NPs), due to their high surface area for conjugation and biodegradability, are attractive as a carrier for stabilization and sustained delivery of therapeutic agents and protein drugs. As described further herein, the release characteristics of model molecules encapsulated in NPs produced from poly(lactide-co-glycolide fumarate) (PLGF) macromer was compared with those conjugated to NPs produced from succinimide (NHS)-terminated PLGF-NHS macromer. Poly(lactide fumarate) (PLAF), (PLGF) and poly(lactide-co-ethylene oxide fumarate) (PLEOF) macromers were synthesized by condensation polymerization. The hydroxyl end-groups of PLAF and PLGF macromers were reacted with N,N′-Disuccinimidyl carbonate (DSC) to produce succinimide-terminated PLAF-NHS and PLGF-NHS macromers. The macromers were self-assembled by dialysis to form NPs. The amphiphilic PLEOF macromer was used as the surfactant to stabilize the NPs in the process of self-assembly. 1-(2-pyridylazo)-2-naphthol (PAN) was used as a model small molecule for encapsulation in PLAF or PLGF NPs and bovine serum albumin (BSA) was used as a model protein for conjugation to PLAF-NHS and PLGF-NHS NPs. The release profile of the encapsulated PAN from PLAF and PLGF NPs was non-linear and consisted of a burst release followed by a period of sustained release. The release profile for BSA, conjugated to PLAF-NHS and PLGF-NHS NPs, was linear up to complete degradation of the NPs. PLGF and PLAF NPs degraded in 15 and 28 days, respectively, while PLGF-NHS and PLAF-NHS NPs degraded in 25 and 38 days, which demonstrated that the release was dominated by erosion of the matrix. PLAF-NHS and PLGF-NHS NPs are potentially useful as a carrier for sustained in-situ release of protein drugs.

### Material

L-lactide (LA; >99.5% purity) and glycolide (GL; >99.0% purity) monomers were obtained from Ortec (Easley, S.C.) and Boehringer Chemicals (Ingelheim, Germany), respectively. Dichloromethane (DCM), DMF, diethyl ether, and hexane were purchased from Acros (Fairfield, Ohio). Calcium hydride, Poly(ethylene glycol) (PEG, nominal molecular weights 3.4 kDa), triethylamine (TEA), Tin (II) 2-ethylhexanoate (TOC), and DMSO were purchased from Aldrich (Sigma-Aldrich, St. Louis, Mo.). Fumaryl chloride (FC) was obtained from Aldrich and distilled at 161° C. prior to use. Diethylene glycol (DEG; >99% purity) was purchased from Fisher (Pittsburgh, Pa). Dulbecco's phosphate-buffer saline (PBS) was purchased from GIBCO BRL (Grand Island, N.Y.). Spectro/Por dialysis tube (molecular weight cutoff 3.5 kDa) was purchased from Spectrum Laboratories (Rancho Dominguez, Calif.). PAN and BSA (IgG and protease free), as surrogate molecules for release studies, were obtained from Acros and Jackson ImmunoResearch Laboratories (West Grove, Pa.), respectively. N,N′-Disuccinimidyl carbonate (DSC) was obtained from Novabiochem (EMD Biosciences, San Diego, Calif.) and Ninhydrin reagent (2% solution in DMSO) was purchased from Sigma-Aldrich. DCM was purified by distillation over calcium hydride. All other solvents were reagent grade and were used as received without further purification.

### Macromer Synthesis

PLAF and PLGF (50/50 wt % lactide and glycolide) macromers were synthesized by condensation polymerization of ultra low molecular weight poly(lactide) (ULMW PLA) and poly(lactide-co-glycolide) (ULMW PLGA) with FC. The amphiphilic PLEOF macromer was synthesized by condensation polymerization of ULMW PLA and PEG with FC. The ULMW PLA and PLGA were synthesized by ring opening polymerization of LA and/or GL monomers with DEG as the initiator and TOC as the polymerization catalyst as described Jabbari, E. and X. Z. He, Synthesis and characterization of bioresorbable in situ crosslinkable ultra low molecular weight poly(lactide) macromer. Journal of Materials Science-Materials in Medicine, 2008. 19(1): p. 311-318, incorporated by reference herein. LA and GL monomers were dried under vacuum at 40° C. for at least 12 h before the reaction. Briefly, 50 g LA and 40 g GL were placed in a three-neck flask, equipped with an overhead stirrer, under a stream of nitrogen. After melting the monomers by gradually increasing temperature to 130° C., 5.0 ml DEG and 5.5 ml TOC were added to the flask with stirring and reaction was continued for 12 h at 130° C. After the reaction, unreacted monomers and DEG were removed under vacuum (<1 mm Hg) at 140° C. for at least 6 h. The product was dissolved in DCM, precipitated in ether to remove the high molecular weight fraction. The ether was removed by rotary evaporation and the polymer was re-dissolved in DCM and precipitated twice in hexane. The fractionated ULMW PLA or PLGA was dried under vacuum (<5 mmHg and 40° C.) to remove any residual solvent and stored at −20° C. The polymers were characterized by ¹H-NMR and GPC [41]. M̅ₙ̅, M̅_̅{̅w̅}̅, and polydispersity index (PI) of the ULMW PLA macromer was 1450 Da, 1730 Da, and 1.2, respectively. M̅ₙ̅, M̅_̅{̅w̅}̅, and PI of the ULMW PLGA was 1660 Da, 2150 Da, and 1.3, respectively.

In the next step, PLAF or PLGF was synthesized by condensation polymerization of ULMW PLA or PLGA, respectively, with FC as described in He, X., J. Ma, A. Mercado, W. Xu, and E. Jabbari, Cytotoxicity of paclitaxel in biodegradable self-assembled core-shell poly(lactide-co-glycolide ethylene oxide fumarate) nanoparticles. Pharmaceutical Research, 2008: p. PMID: 18196205, Jabbari, E. and X. He, Synthesis and material properties of functionalized lactide oligomers as in situ crosslinkable scaffolds for tissue regeneration. Polymer Preprints, 2006. 47(2): p. 353-354, Jabbari, E., X. He, M. Valarmathi, A. Sarvestani, and W. Xu, Material properties and bone marrow stromal cells response to in situ crosslinkable rgd-functionlized lactide-co-glycolide scaffolds. Journal Biomedical Materials Research Part A, 2008. in Press, all incorporated by reference herein. Similarly, PLEOF was synthesized by reacting ULMW PLA and PEG with FC as described in Sarvestani, A. S., X. Z. He, and E. Jabbari, Viscoelastic characterization and modeling of gelation kinetics of injectable in situ cross-linkable poly(lactide-co-ethylene oxide-co-fumarate) hydrogels. Biomacromolecules, 2007. 8(2): p. 406-415, Sarvestani, A. S., W. J. Xu, X. Z. He, and E. Jabbari, Gelation and degradation characteristics of in situ photo-crosslinked poly(L-lactid-co-ethylene oxide-co-fumarate) hydrogels. Polymer, 2007. 48(24): p. 7113-7120, He, X. Z. and E. Jabbari, Material properties and cytocompatibility of injectable MMP degradable poly(lactide ethylene oxide fumarate) hydrogel as a carrier for marrow stromal cells. Biomacromolecules, 2007. 8(3): p. 780-792, all incorporated be reference herein. TEA was used as the acid scavenger. The molar ratios of FC:(PEG+PLA) and TEA:(PEG+PLA) were 0.9:1.0 and 1.8:1.0, respectively. In a typical reaction, 2.0 g PEG and 18.0 g PLA were dried by azeotropic distillation with toluene and dissolved in 150 ml DCM under dry nitrogen atmosphere in a three-neck reaction flask. After cooling to 5° C., 1.46 ml FC and 3.65 ml TEA, each dissolved in 60 ml DCM, were added drop-wise to the reaction with stirring. The reaction continued for 6 h on ice followed by 12 h under ambient conditions. After completion of the reaction, solvent was removed by rotary evaporation and residue was dissolved in 100 ml anhydrous ethyl acetate to precipitate the by-product triethylamine hydrochloride and the salt was removed by filtration. Ethyl acetate was removed by rotary evaporation, the product was dissolved in DCM, and precipitated twice in cold ethyl ether. The polymer was dried in vacuum (<5 mmHg) for at least 12 h and stored at −20° C. PLAF and PLGF macromers were synthesized using a similar procedure. The synthesized PLEOF, PLAF and PLGF macromers were characterized by ¹H-NMR and GPC.

### Macromer Modification With Disuccinimidyl Carbonate

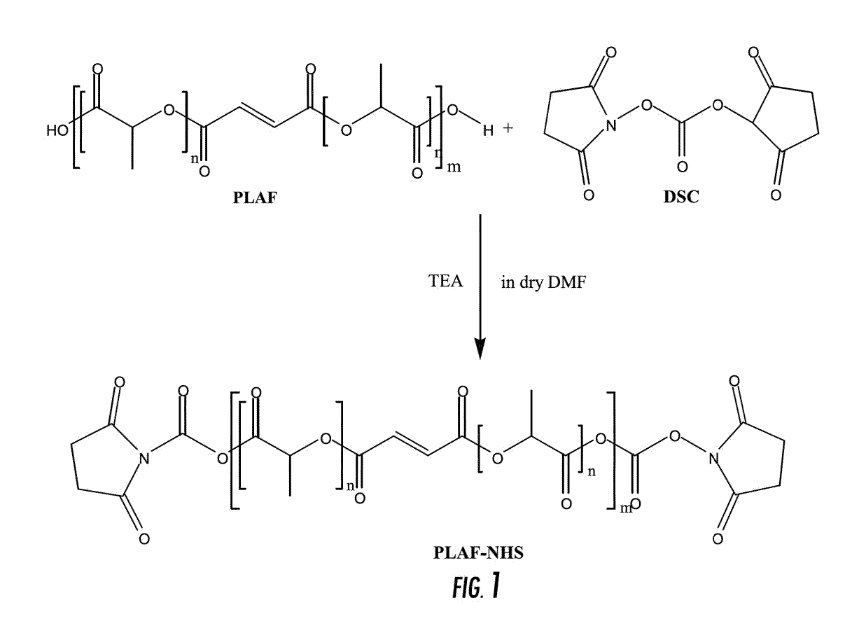

Succinimide-terminated macromers (PLAF-NHS or PLGF-NHS) were produced by reacting the hydroxyl end-groups of the PLAF or PLGF macromers with the carbonate group of DSC, as shown in the schematic diagram of FIG. 1, using a procedure as described in Morpurgo, M., E. A. Bayer, and M. Wilchek, N-hydroxysuccinimide carbonates and carbamates are useful reactive reagents for coupling ligands to lysines on proteins. Journal of Biochemical and Biophysical Methods, 1999. 38(1): p. 17-28, incorporated by reference herein, with modification. Briefly, 800 mg PLAF or PLGF and 26 mg DSC were mixed in 15 ml DMF in a reaction flask. After purging the reaction mixture with nitrogen, 40 μL TEA was added with stirring and the reaction flask was covered with aluminum foil. The reaction was allowed to continue for 8 h at ambient conditions. The resulting mixture was precipitated in ether and the product was separated by filtration. The product was redissolved in DCM, precipitated twice in ether, dried in vacuum, and stored at −20° C. The succinimide terminated macromer was characterized by ¹H-NMR.

### Characterization of Macromers

The chemical structure of the synthesized macromers was characterized by a Varian Mercury-300 ¹H-NMR (Varian, Palo Alto, Calif.) at ambient conditions with 0.17 Hz resolution. The macromer was dissolved in deuterated chloroform (Aldrich, 99.8 atom % deuterated) at a concentration of 50 mg/ml, and 1% v/v trimethylsilane (TMS; Aldrich) was used as the internal standard. The molecular weight distribution of the macromers was measured by GPC. Measurements were carried out with a Waters 717 Plus Autosampler

GPC system (Waters, Milford, Mass.) connected to a model 616 HPLC pump, model 600S controller, and a model 410 refractive index detector. The columns consisted of a styragel HT guard column (7.8×300 mm, Waters) in series with a styragel HR 4E column (7.8×300 mm, Waters) heated to 37° C. in a column heater. The Empower software (Waters) was used for data analysis and determination of M̅ₙ̅, M̅_̅{̅w̅}̅, and PDI. The sample (20 μl), with a concentration of 10 mg/ml in tetrahydrofuran (THF; Sigma-Aldrich), was eluted at a flow rate of 1 ml/min. Monodisperse polystyrene standards (Waters) with peak molecular weights (Mₚ) ranging from 0.58 to 143.4 kDa and PDI of less than 1.1 were used to construct the calibration curve.

### NPs Formation by Self-Assembly of the Macromers

Mixtures of PLAF-NHS (or PLGF-NHS) and PLEOF macromers in DMF/DMSO solvent mixture were self-assembled into NPs by dialysis against water as described in Gao, H., Y. N. Wang, Y. G. Fan, and J. B. Ma, Conjugates of poly(DL-lactide-co-glycolide) on amino cyclodextrins and their nanoparticles as protein delivery system. Journal of Biomedical Materials Research Part A, 2007. 80A(1): p. 111-122, Xie, J. W. and C. H. Wang, Self-assembled biodegradable nanoparticles developed by direct dialysis for the delivery of paclitaxel. Pharmaceutical Research, 2005. 22(12): p. 2079-2090, both incorporated by reference herein. Briefly, 45 mg PLAF-NHS (or PLGF-NHS) and 5 mg PLEOF macromers were dissolved in a solution of 1 ml DMF and 8 ml of DMSO. If PAN was used as the surrogate molecule for release experiments, 3.2 mg PAN (6% by weight of macromers) in 1 ml DMSO was added to the macromer solution. The solution was filtered with a 0.2 μm filter (Whatman autovial syringeless filter with a PTFE membrane; Fisher) and loaded in the dialysis tube (molecular cutoff: 3.5 kDa) and dialyzed against distilled deionized (DI) water. The solution was dialyzed for 24 h with change of dialysis buffer every 4 h until DMSO and DMF were completely removed. Then, the suspension containing the self-assembled NPs was collected from the dialysis tube and freeze-dried to obtain a free flowing powder. The NPs suspension was used directly for particle size measurements before freeze-drying. PAN-free NPs were prepared using a similar procedure without the addition of PAN.

### Conjugating BSA to Succinimide Terminated NPs

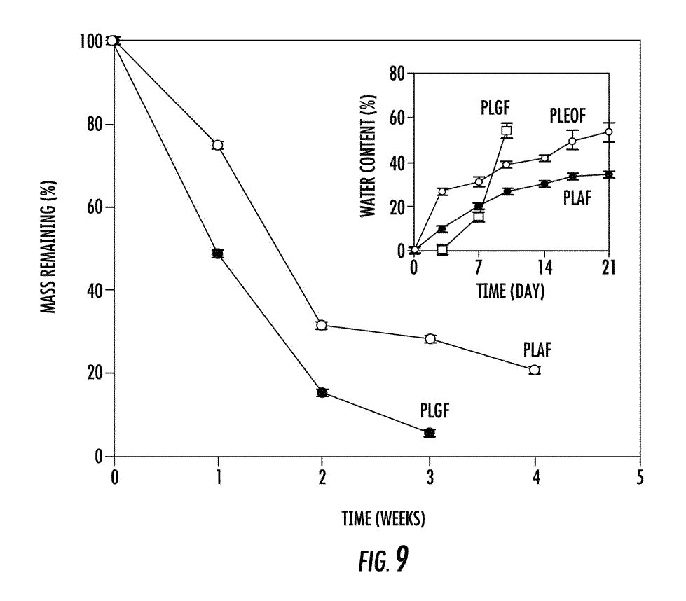

NPs were prepared by dialysis of PLAF-NHS/PLEOF or PLGF-NHS/PLEOF macromers in DMF/DMSO mixture against DI water, as described in section 2.5. After dialysis, 1 ml of the suspension (2.5 mg NPs/ml suspension) was centrifuged at 18,350 rcf (15,000 rpm) for 10 min, the supernatant was decanted, and the precipitated NPs were re-suspended in 0.5 ml PBS by sonication for 1 min with a 3-mm probe connected to an Ultrasonic Processor (Model CP-130PB-1, continuous mode, Cole-Parmer Instruments, Vernon Hills, Ill.) with a power and frequency of 10 Watts and 20 kHz, respectively. After sonication, a noticeable change in particle size distribution of the NPs was not observed before and after centrifugation, as measured by dynamic light scattering. Next, 0.5 ml BSA in PBS solution (20 mg BSA/ml) was added to the suspension and mixed. The BSA was allowed to react with the succinimide terminated NPs under ambient conditions for 12 h. After reaction, the suspension was dialyzed against PBS to remove the by-product, N-hydroxy succinimide. Based on the core-shell model for the structure of NPs (see FIG. 8), the shell volume can be as much as 85% of the total volume of the NPs and the shell is composed of low molecular weight fractions of PLAF/PLGF macromers and PLEOF. Therefore, assuming all PLEOF macromers were in the shell, at least 60% of the low molecular weight fractions (higher density of chain-end succinimide groups compare to high molecular weight fractions) PLGF was also in the shell. To determine conjugation efficiency, the dialyzed suspension was centrifuged at 18,350 rcf for 10 min, and the amount of BSA in the supernatant was measured with Ninhydrin reagent as described in section 2.10. For release experiments, the dialyzed suspension was centrifuged at 18,350 rcf for 10 min, the supernatant was removed, and the NPs were washed with 1 ml PBS to remove the unconjugated BSA. The amount of BSA in the PBS wash was measured with Ninhydrin reagent. This process was repeated (at least three times) until the concentration of BSA in the PBS wash was less than the detection limit of 0.08 mg/ml, corresponding to absorbance of 0.09. After washing, the amount of unconjugated BSA in the NPs suspension was <3% of the initial amount (0.08 mg versus 2.5 mg).

### Characterization of NPs

The morphology and size distribution of the NPs was examined using a JSM-5400 scanning electron microscope (JOEL, Japan) at an accelerating voltage of 20 KeV. Freeze-dried NPs were placed on a graphite surface and coated with gold using an Ion Sputter Coater (JEOL, JFC-1100) at 20 mA for 1 min. The size distribution of NPs was measured by dynamic light scattering with a NICOMP Submicron Particle Sizer (Autodilute Model 370, NICOMP Particle Sizing Systems, Santa Barbara, Calif.). 500 μl of the diluted suspension was added to a culture tube and placed in the instrument cell holder. The scattered light intensity was inverted to size distribution by inverse Laplace transform using the CW370 software (NICOMP Particle Sizing Systems).

### Degradation of NPs

Degradation of the NPs was followed by measuring their particle size and mass loss as a function of incubation time. PLAF-NHS/PLEOF or PLGF-NHS/PLEOF NPs were prepared by dialysis of a solution of the macromers in DMF/DMSO solvent mixture against DI water, as described in section 2.5. After self-assembly, the NPs suspension was centrifuged at 18,350 rcf for 10 min, the supernatant was decanted to remove the unassembled macromers, and the NPs were re-suspended in PBS. For degradation experiments, 50 mg NPs were suspended in 1 ml PBS and the suspensions were incubated at 37° C. until complete degradation (no mass remaining or NPs not detectable by dynamic light scattering). At each time point, the size distribution of the NPs was measured by dynamic light scattering, as described herein. Next, samples were freeze-dried and mass of the dried powder was measured. The fraction of mass remaining was determined by dividing the dried mass at time t by the initial mass at time zero.

To determine water content, disk-shape samples of PLAF, PLGF, and PLEOF (750 μm thickness×5 mm diameter) were incubated in 5 ml PBS at 37° C. and the swelling medium was changed every 24 h. At each time point, the weight of the sample, Wₜ, was measured and the water content was determined by (Wₜ-W_{d})/Wₜ, where W_{d} was the dry sample weight. To measure W_{d}, samples were washed with DI water to remove excess electrolytes and dried at ambient conditions for 12 h, followed by drying in vacuum at 40° C. for 1 h.

### Loading Efficiency and Release Kinetics of PAN-Loaded NPs

Since the molecular weight cutoff of the dialysis membrane was much higher than the molecular weight of PAN (238 Da for PAN versus 3500 Da cutoff for membrane), the unencapsulated PAN was removed in the process of NPs formation by dialysis. To determine loading efficiency, PAN-loaded NPs were prepared by dialysis, as described herein, and freeze-dried. Next, the dried NPs were dissolved in DMF and absorbance was measured at 615 nm with a plate reader (Synergy HT, Bio-Tek, Winooski, Vt.). The measured absorbance was subtracted from the absorbance of the PAN-free NPs. The measured absorbance was related to PAN concentration using a calibration curve constructed from absorbance of solutions with known PAN concentration (in the linear range of the detector). Loading efficiency was determined by dividing the amount of encapsulated PAN to the initial amount. For determination of release kinetics, 1 mg PAN-loaded NPs were placed in microcentrifuge tubes and incubated with 1 ml PBS (pH 7.4) at 37° C. with orbital shaking Since the present disclosure relates to release mechanism of model compounds from the NPs, the release of PAN and BSA was measured in PBS, in agreement with previous work as described in Lee, J., E. Cho, and K. Cho, Incorporation and release behavior of hydrophobic drug in functionalized poly(D,L-lactide)-block-poly(ethylene oxide) micelles. Journal Controlled Release, 2004 94(2-3): p. 323-35, Katsikogianni, G. and K. Avgoustakis, Poly(lactide-co-glycolide)-methoxy-poly(ethylene glycol) nanoparticles: Drug loading and release properties. Journal of Nanoscience and Nanotechnology, 2006. 6(9-10): p. 3080-3086, Westedt, U., M. Kalinowski, M. Wittmar, T. Merdan, F. Unger, J. Fuchs, S. Schaller, U. Bakowsky, and T. Kissel, Poly (vinyl alcohol)-graft-poly(lactide-co-glycolide) nanoparticles for local delivery of paclitaxel for restenosis treatment. Journal of Controlled Release, 2007. 119(1): p. 41-51, Gao, H., Y. N. Wang, Y. G. Fan, and J. B. Ma, Conjugates of poly(DL-lactide-co-glycolide) on amino cyclodextrins and their nanoparticles as protein delivery system. Journal of Biomedical Materials Research Part A, 2007. 80A(1): p. 111-122, all incorporated by reference herein. At each time interval, the suspension was centrifuged at 18,350 rcf for 10 min, and the supernatant was removed and poured into microvials for analysis. Next, the NPs were resuspended in 1 ml fresh PBS and incubated until the next time interval. For PAN-loaded NPs, the absorbance of the supernatant at 615 nm was measured with a Synergy HT plate reader. The measured absorbance was subtracted from the absorbance of the supernatant of PAN-free NPs incubated for the same duration of time. The measured amount of PAN at time t was divided by the initial amount to determine the fraction of PAN released.

### Conjugation Efficiency and Release Kinetics of BSA-Conjugated NPs

BSA-conjugated NPs were synthesized by the reaction of BSA with succinimide terminated NPs, as described herein. After conjugation, the suspension was dialyzed against PBS to remove the by-product, N-hydroxy succinimide. Since the molecular weight cutoff of the dialysis membrane was much lower than the molecular weight of BSA (67 kDa for BSA versus 3500 Da cutoff for membrane), the unconjugated BSA was not removed by dialysis. To determine conjugation efficiency, the dialyzed suspension was centrifuged at 18,350 rcf for 10 min, and the supernatant was poured into microvials for analysis with Ninhydrin reagent as described in He, X. Z. and E. Jabbari, Solid-phase synthesis of reactive peptide crosslinker by selective deprotection. Protein and Peptide Letters, 2006. 13(7): p. 515-518, incorporated by reference herein. For analysis of the free BSA, 200 μl of the supernatant was incubated at 37° C. for 12 hr and then 50 μl of Ninhydrin reagent (Sigma-Aldrich) was added. After mixing, the sample was heated to 120° C. for 5 min and the absorbance was measured at 570 nm with a Synergy HT plate reader. The measured absorbance was related to the concentration using a calibration curve constructed from the absorbance of solutions with known concentrations of BSA. Conjugation efficiency was determined by dividing the amount of free BSA by the initial amount of BSA. For determination of release kinetics, 1 mg BSA-loaded NPs were placed in microcentrifuge tubes and incubated with 1 ml PBS (pH 7.4) at 37° C. with orbital shaking At each time interval, the suspension was centrifuged at 18,350 rcf for 10 min, and the supernatant was removed and poured into microvials for analysis. Next, the NPs were resuspended in 1 ml fresh PBS and incubated until the next time interval. At each time point, the amount of released BSA in the supernatant was measured with Ninhydrin reagent as described above.

### Results and Discussion

### Macromer Characterization

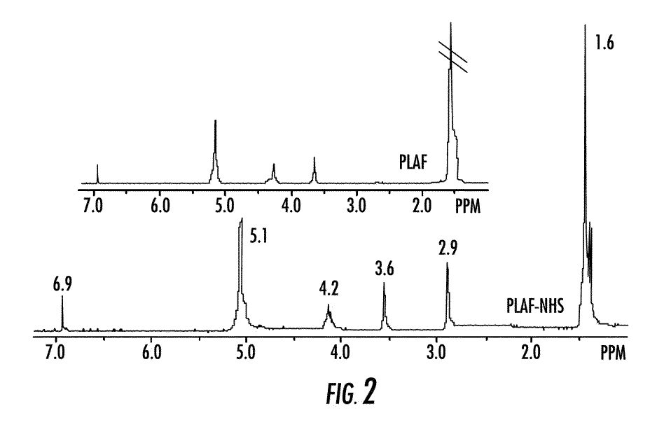

¹H-NMR spectra of PLAF and PLAF-NHS are shown in FIG. 2. The doublet chemical shift with peak position at 1.6 ppm, two triplets with peak positions at 3.6 and 4.2 ppm, and a quartet with peak position at 5.1 ppm were attributed to the methyl hydrogens (—CH₃) of the lactide repeat unit, the methylene hydrogens of the DEG initiator, and the methine hydrogen of the lactide. The singlet shifts at 6.90 and 6.95 ppm were attributed to the methine hydrogens of the fumarate in the middle of the chain (—OOC—CH═CH—COO—) and at chain ends (—OOC—CH═CH—COOH), respectively. The additional chemical shift in the spectrum of PLAF-NHS with peak position at 2.9 ppm was attributed to the methylene hydrogens of the succinimide end-groups. The presence of a chemical shift at 2.9 ppm in the NMR spectrum and the presence of absorption bands at 2900 and 1780 cm⁻¹ due to methyl and carbonyl vibrations in the FTIR spectra (data not shown), confirmed the incorporation of succinimide group to PLAF chain ends. The ratio of the chemical shifts centered at 3.6 and 4.2 ppm [methylene groups on DEG initiator attached to ether (—CH₂—O—CH₂—) and ester groups of the PLA (—CH₂—OOC—), respectively; total of 8 hydrogens per chain] to that at 2.9 ppm (methylene groups of the succinimide end groups; total of 4 hydrogen] was proportional to the number of succinimde end groups per chain. This ratio, on average, was 1.4 succinimde groups/chain for PLAF-NHS and PLGF-NHS. M̅ₙ̅, M̅_̅{̅w̅}̅, and PI of the synthesized PLAF was 4.5 kDa, 8.6 kDa, and 1.9, respectively; those of PLGF was 5.2 kDa, 10.9 kDa, and 2.1; and those of PLEOF was 9.7 kDa, 15.4 kDa, and 1.6. Attachment of succinimide end-groups to PLAF macromer resulted in an increase in M̅ₙ̅ from 4.5 to 4.7 kDa and a slight increase in M̅_̅{̅w̅}̅ from 8.61 to 8.63 kDa (PDI decreased slightly from 1.91 to 1.83). Similar results were obtained for PLGA-NHS.

### Size Distribution of Self-Assembled NPs

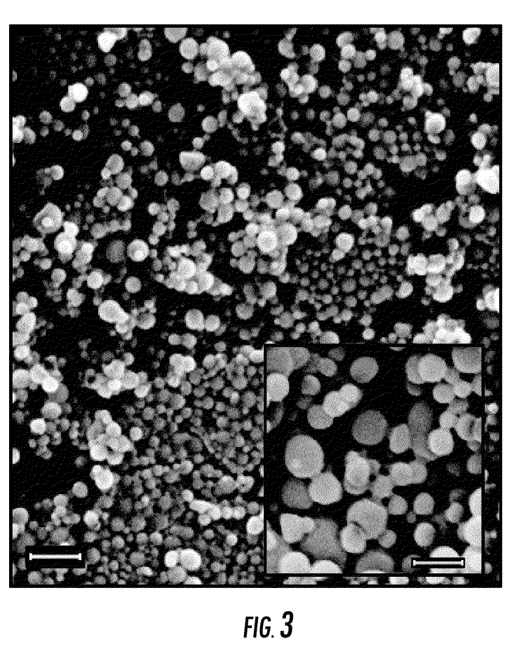

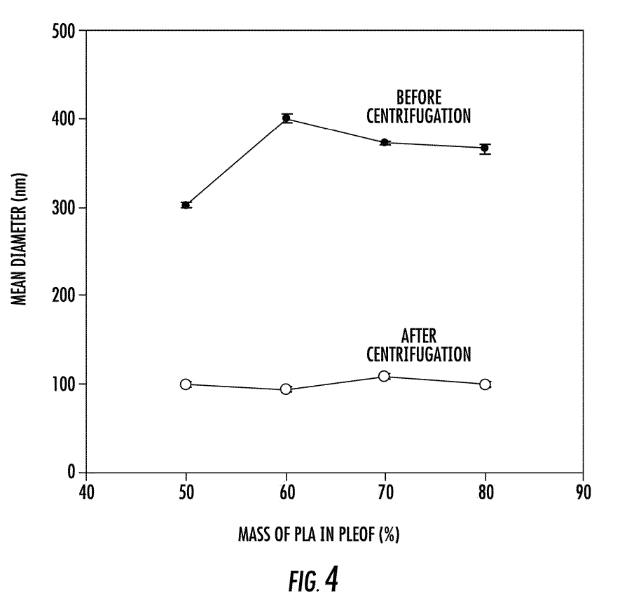

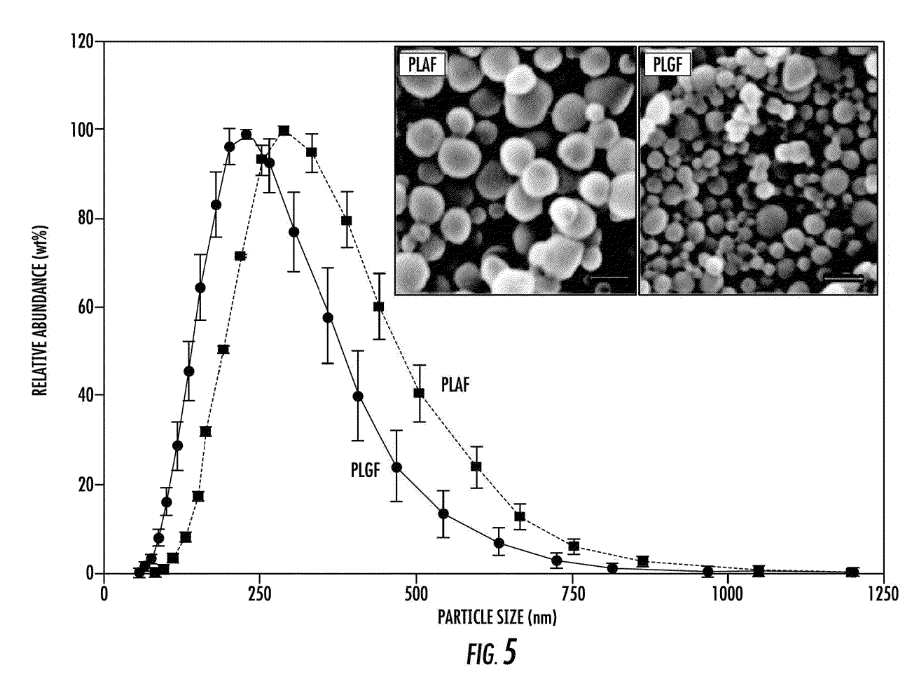

Blends of PLAF/PLEOF or PLGF/PLEOF, with or without succinimide termination, (90% PLAF or PLGF and 10% PLEOF) were dissolved in DMF/DMSO solvent mixture and the polymer solutions were dialyzed against DI water to form self-assembled NPs. A typical morphology of the NPs is shown in the SEM image of FIG. 3. The NPs had spherical shape and smooth surface morphology with size distribution in the 50-500 nm range, as shown in the insert image of FIG. 3. Particles formed by dialysis of PLAF or PLGF macromers without the addition of PLEOF had sizes >1 μm and non-spherical in shape (images not shown). PLEOF is an amphiphilic macromer with hydrophilic PEG units and hydrophobic PLA units. The smaller particle size of NPs prepared with PLAF/PLEOF or PLGF/PLEOF demonstrated that the amphiphilic PLEOF macromer functioned as a surface active agent in the process of particle formation. The effect of PLA fraction in PLEOF macromer on the size of PLAF/PLEOF NPs is shown in FIG. 4. The average diameter of PLAF NPs produced with 80, 70, 60, and 50 wt % PLA in PLEOF changed from 365±6 to 373±3, 400±5, and 302±3 nm, respectively. PLAF NPs with 60% PLA in PLEOF had the largest size while those with 50% PLA had the smallest size. Centrifugation of the NPs colloidal suspension after dialysis significantly reduced the size of the NPs, as shown in FIG. 4. After dialysis, the NPs suspension was centrifuged at 82 rcf, (1000 rpm) the precipitate was removed, and the size distribution of the NPs in the supernatant was measured. PLAF NPs in the supernatant with 80, 70, 60, and 50 wt % PLA in PLEOF had average diameter of 100±2, 108±3, 93±2, and 100±3 nm, respectively, as shown in FIG. 4. These results demonstrate that dialysis, followed by centrifugation, can be used to produce NPs with sizes ≦100 nm for uptake by tumor vasculature and targeted tumor delivery. Tumor blood vessels have several abnormalities compared with normal vessels resulting in enhanced permeation and retention effect (EPR effect). The pore cutoff size of most tumor models is 200-400 nm and NPs with diameter ≦100 nm are selectively taken up by tumor tissues. Therefore, PLAF and PLGF NPs provide the opportunity to selectively target the tumor tissue over normal tissue. The PLEOF with PLA and PEG fractions of 70 and 30 wt % was used in preparation of PLAF and PLGF NPs for experiments to measure degradation and release characteristics. The size distribution of PLAF and PLGF NPs (90% PLAF or PLGF and 10% PLEOF) are compared in FIG. 5. The average size of PLAF and PLGF NPs was 325 and 250 nm, respectively, with breadth of distribution (one standard deviation from the mean) of 135 and 115 nm. PLAF-NHS and PLGF-NHS NPs had similar morphologies, as shown in the SEM images in the insert of FIG. 5. The BSA conjugated NPs were imaged with atomic force microscopy (NanoScope IIIA MultiMode AFM; Veeco Instruments, Plainview, N.Y.). Si tips with a resonance frequency of approximately 300 kHz and a spring constant of about 40 N m⁻¹ were used, and the scan rate was 1.0 Hz. However, due to polydispersity in the size of NPs, the images did not provide insight into the nature of interactions between the model molecules and NPs. Nano Differential Scanning Calorimetry (Nano DSC; TA Instruments, New Castle, Del.), which measures the amount of heat absorbed/released as the sample is heated/cooled on the nanowatt scale, is potentially useful for studying the conformation of proteins. This technique can be used to study the conformation of BSA conjugated to PLGF-PLEOF NPs.

### Degradation Characteristics of NPs

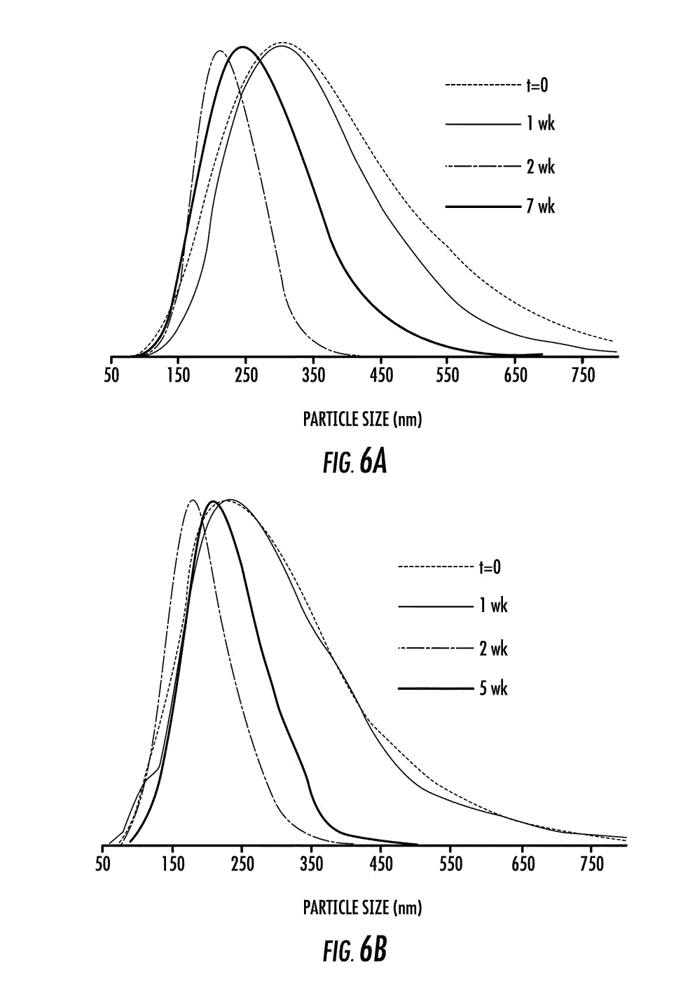

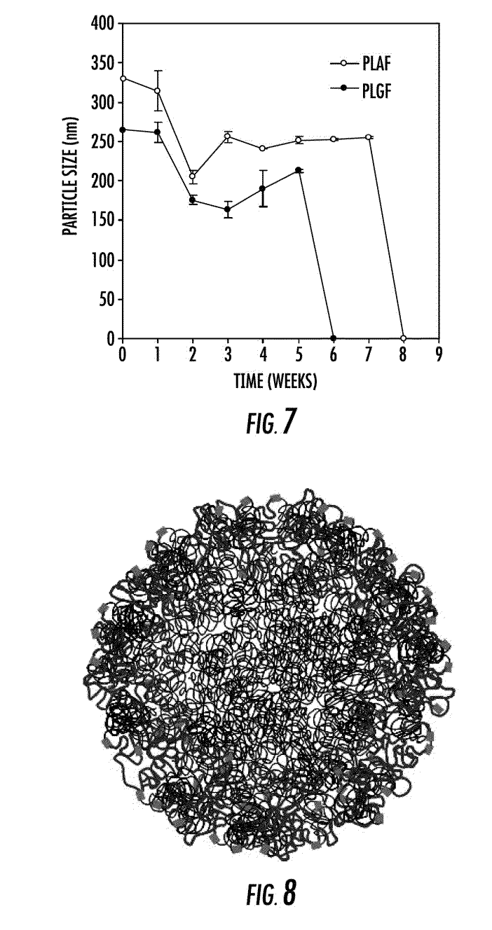

Degradation of the NPs was followed by measuring their particle size and mass loss with time. FIGS. 6(a) and 6(b) show the changes in size distribution of PLAF and PLGF NPs (90% PLAF or PLGF and 10% PLEOF), respectively, as a function of time. There was a slight decrease in size in the first week followed by a significant decrease in average size and size distribution after 2 weeks for PLAF and PLGF NPs. This was followed by an increase in average size and distribution after 7 weeks for PLAF and 5 weeks for PLGF NPs, respectively. For example, for PLGF NPs, the average size decreased slightly from 262 to 260 nm after one week, then decreased significantly to 183 nm after 2 weeks, and then increased to 216 nm after 5 weeks. The breadth of the distribution followed the same trend for PLAF and PLGF NPs. The change in average particle size with incubation time for PLAF and PLGF NPs (90% PLAF or PLGF and 10% PLEOF) is shown in FIG. 7. PLAF and PLGF NPs showed similar trends in particle size with time. For PLAF and PLGF NPs, particle size decreased significantly after 2 weeks and then increased slightly before degrading completely after 6 weeks for PLGF and 8 weeks for PLAF.

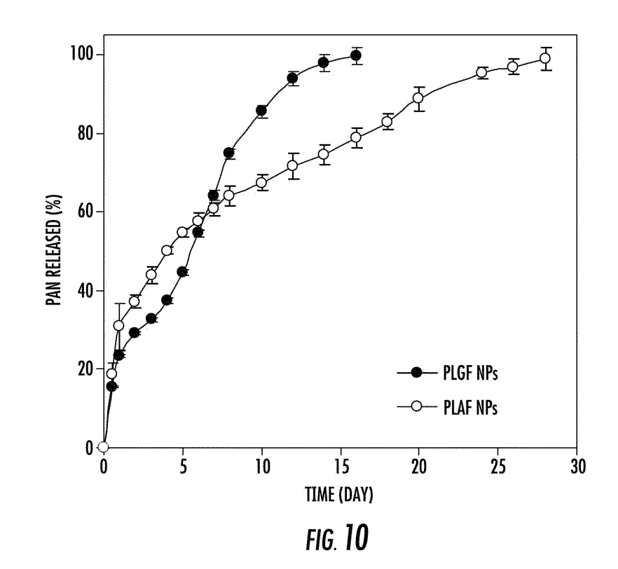

Based on these results, a core-shell model is proposed for the structure of NPs, as shown schematically in FIG. 8. The area in the center with dark lines (PLGF chains) is the core and that with dark (PLGF chains) and blue lines (PLEOF chains) is the shell. The higher molecular weight fractions in the PLGF macromer are more hydrophobic than the low molecular weight fractions because the hydroxyl and carboxylic acid chain ends in the former contribute less than the latter to the overall hydrophilicity of the macromer. Therefore, in the process of dialysis, the higher molecular weight fractions undergo phase separation first to form the core of the NPs followed by the low molecular weight PLGF fractions and PLEOF which form the NPs' shell. The relatively fast reduction in NPs size in the first two weeks can be explained by dissolution/degradation of the shell with higher fraction of PLEOF followed by the relatively slower (especially in the case of PLAF) degradation (with concurrent swelling and increase in NPs size) of the hydrophobic core. The mass loss of PLAF and PLGF NPs as a function of time is shown in FIG. 9. The mass loss data in FIG. 9 shows that 69% and 85% of the mass of PLAF and PLGF NPs, respectively, mostly due to dissolution/degradation of the shell, is lost in the first 2 weeks of incubation. The core-shell model in FIG. 8 can be used to explain the relatively large fractional mass loss in the first two weeks of incubation. The relative core radius and shell thickness in FIG. 8 are 1.0 and 0.90, respectively, resulting in relative core and shell volumes of 1.0 and 6.9. Therefore, based on the dimensions in FIG. 8, the volume of the shell is 6.9 times that of the core. Since mass loss is proportional to the volume (assuming constant density), the dissolution/degradation of the shell constitutes 87% of the mass loss. The water content of PLAF, PLGF, and PLEOF disks with incubation time is also shown in the insert of FIG. 9. The water content of the NPs increased in week 1 and 2 with concurrent dissolution/degradation of the shell (with higher fraction of hydrophilic PLEOF) followed by slow degradation of the core.

### Release Characteristics

PLAF-NHS and PLGF-NHS NPs provide the opportunity to conjugate, as opposed to encapsulate, bioactive proteins to the NPs via the reaction of terminal succinimide groups with the amine groups on proteins. PLAF or PLGF macromers, without termination with reactive succinimide groups, were used for encapsulation of the model drug PAN (238 Da) and release was measured by monitoring absorbance at 630 nm. PLAF-NHS or PLGF-NHS macromers were used for conjugating the model protein drug BSA (67 kDa) to the NPs and release was measured by monitoring the amine concentration in the release medium using Ninhydrin reagent.

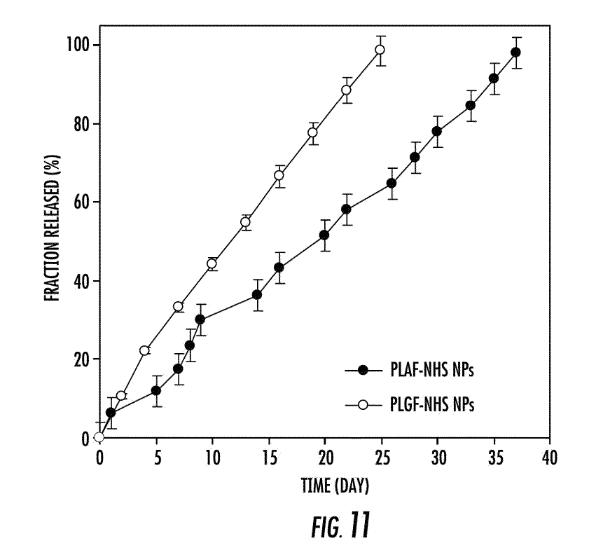

The release characteristics of the encapsulated PAN from PLAF and PLGF NPs in FIG. 10 are compared with that of conjugated BSA to PLAF-NHS and PLGF-NHS NPs in FIG. 11, respectively. The average PAN encapsulation efficiency in PLAF and PLGF NPs was 63±10%. The encapsulation efficiency was independent of the macromer (PLAF or PLGF) but it decreased significantly with increasing PAN loading. The release profile for encapsulated PAN was non-linear and consisted of a burst release in the first 24 h followed by a period of sustained release. The burst release in the first 24 h can be attributed to the partial segregation of PAN to the surface in the process of centrifugation, freeze-drying, and resuspension of the NPs in PBS for release experiments. The more hydrophobic PLAF NPs released PAN at a relatively constant rate in 28 days while the less hydrophobic PLGF NPs displayed a slow release regime between days 1-4 followed by a period of fast release between days 4-15. PLGF NPs, due to lower hydrophobicity and faster degradation, released the encapsulated PAN in 15 days, while PLAF NPs released their content in 28 days. PLGF and PLAF NPs completely degraded in 15 and 28 days (no precipitate was obtained when suspensions were centrifuged at 18,350 rcf) which demonstrated that the release was dominated by matrix erosion. It should be noted that the hydrophilic end groups of PLGF and PLEOF macromers provide a driving force for diffusion of water in the NPs, resulting in the release of the small molecules from the NPs by diffusion and erosion.

The release characteristic of BSA conjugated to PLAF-NHS and PLGF-NHS NPs is shown in FIG. 11. The average size of the NPs increased from 180 to 240 nm after BSA conjugation and breadth of the distribution increased from 50 to 70 nm. The average BSA conjugation efficiency in PLAF-NHS and PLGF-NHS NPs was 61±20%. The release profile for conjugated BSA for both PLAF-NHS and PLGF-NHS was linear up to complete degradation time of the NPs. Similar to PLAF and PLGF NPs, the more hydrophobic PLAF-NHS NPs released BSA at a constant rate of 2.5% of the conjugated BSA/day in 38 days while the less hydrophobic PLGF-NHS NPs displayed a faster constant release rate of 3.7%/day in 25 days. PLGF-NHS and PLAF-NHS NPs completely degraded in 25 and 38 days which demonstrated that the release was dominated by hydrolytic degradation and erosion of the matrix. The reaction between PLAF/PLGF macromer and DSC converts the hydrophilic hydroxyl and carboxylic acid end-groups of the macromer to hydrophobic succinimide groups. Therefore, NPs produced from succinimide-terminated macromers are significantly more hydrophobic than those produced from PLAF/PLGF. As a result, PLAF-NHS/PLGF-NHS NPs have slower degradation rate and slower release of BSA compared to encapsulated PAN from PLAF/PLGF NPs. Diffusion also plays a role in the release from the NPs, because the hydrophilic end groups of PLGF and PLEOF macromers provide a driving force for diffusion of water in the NPs, resulting in the release of small molecules by diffusion.

The stability and activity of the released protein from the NPs was assessed with recombinant human bone morphogenetic protein-2 (rhBMP-2). To test the stability of the protein, 100 μg/ml rhBMP-2 was incubated in PBS containing 5 wt % PLEOF. Samples were removed with time and the enzymatically active concentration of rhBMP-2 was measured by enzyme-linked immunosorbent assay (ELISA) using a BMP Quantikine kit (R&D Systems, Minneapolis, Minn.). The relative absorbance measured after 1, 15, and 30 days was 100.0±7.5, 102.0±7.6, and 103.0±7.7, respectively. These results demonstrate that the protein was stable for 30 days in the assay. To test the activity of the released protein, rhBMP-2 was conjugated to PLAF-NHS NPs, and the suspension was dialyzed against PBS to remove the by-product, N-hydroxy succinimide. 1 mg rhBMP-2 conjugated NPs were placed in microcentrifuge tubes and incubated with 1 ml PBS. At each time point, the suspension was centrifuged, supernatant was removed, and the enzymatically active concentration of rhBMP-2 was measured by ELISA. As incubation time was increased from 1 to 5, 10, 21, 35, and 42 days, the percent of the released protein increased from 2.9±0.2 to 13.8±0.3, 26.1±0.9, 38.6±0.7, 45.8±0.9, and 48.7±0.7%, respectively, and the NPs had completely degraded after 42 days. After complete degradation of the NPs, about 50% of the released protein was enzymatically active. These results suggest that about 50% of the rhBMP-2 protein is deactivated in the process of conjugation or later in the process of degradation of the NPs to release the conjugated protein.

While the release of model drugs from high molecular weight (40-60 kDa) PLA and PLGA systems is by diffusion through a porous matrix, the release from PLAF-NHS and PLGF-NHS NPs, with or without termination with succinimide groups, is dominated by matrix erosion. Hydrophobic and hydrophilic drugs can be encapsulated in PLGA NPs using single and double emulsion methods and the release characteristics can be tailored to a particular application by varying the lactide/glycolide ratio. However, due to the high polymer molecular weight and its hydrophobicity, drug loading in PLGA NPs is relatively low (especially for hydrophilic drugs) resulting in a significant fraction of burst release and a slow long-term release. Furthermore, non-degradable surfactants are used to stabilize the PLGA NPs which affect the physical and biological properties of the NPs. To facilitate self-assembly and NP formation, PLGA is Pegylated which improves loading of small molecule drugs or the stability of proteins drugs but most of the drug is released from the Pegylated PLGA NPs in the first day of incubation. For example, when Cisplatin was encapsulated in Pegylated PLGA NPs of varying PLGA molecular weights ranging from 7 to 68 kDa and PEG molecular weight of 5 kDa, >70% of the drug was released in the first 24 h followed by a sustained release period of 3-5 days. Heparin-conjugated PLGA NPs have been synthesized for sustained delivery of basic fibroblast growth factor (bFGF) by association of bFGF with heparin conjugated NPs. This method provided sustained delivery of bFGF for up to 4 weeks but it required conjugation of the heparin molecule (molecular weight of 3-15 kDa depending on the activity) to the NPs for association with bFGF. In past experiments, it is noteworthy to mention that the amount of heparin conjugated to the PLGA NPs increased up to 29-fold by using NPs made from lower molecular weight PLGA, as compared to NPs made from higher molecular weight PLGA [61]. PLAF and PLGF macromers in combination with amphiphilic PLEOF self-assemble to form NPs with degradable PLEOF macromer acting as a surfactant to stabilize the NPs. The low molecular weight of PLAF and PLGF macromer ( M̅_̅{̅w̅}̅<10 kDa) and the formation of amorphous NPs after self-assembly (PLAF and PLGF macromers have negligible crystallinity compared to PLGA polymer) improve solubility of small molecule drugs in the NPs. Due to low molecular weight of the macromer (higher density of chain ends compared to PLGA polymer), the chains ends contribute significantly to the hydrophobicity of PLAF and PLGF macromers, which can potentially be used for encapsulation of drugs with different degree of hydrophobicity. The short PLAF and PLGF chains, combined with the contribution of chain ends, make NPs to release the encapsulated drug predominantly by matrix erosion. The erosion rate can be varied for up to 5 weeks by changing the lactide/glycolide ratio or by termination of chain ends with succinimide groups. Furthermore, the high density of chain ends in PLAF and PLGF macromers can increase the amount of the protein drug that can be conjugated to the NPs. The effect of molecular weight of PLAF and PLGF on degradation and release characteristics is in progress and the results will be reported in future communications.

The constant release rate from PLAF-NHS and PLGF-NHS NPs combined with their complete degradation is very attractive for delivery of chemotherapeutic agents to tumor tissues. When the chemotherapeutic agent Paclitaxel was encapsulated in PLGF-PLEOF and PLAF-PLEOF NPs, sustained release of the drug was observed in vitro for 15 and 28 days, respectively. Furthermore, the Apc^{Min/+} mouse (with intestinal tumor) injected with PLGF-PLEOF NPs displayed at least 100 times higher intensity of the NPs in the intestinal region that other organs. Since most tumors lack lymph vessels and higher interstitial fluid pressure than normal tissues, NPs are accumulated in the interstitium which retards their additional uptake, unless the NPs degrade to molecular weights below 50 kDa. The degradable PLGF-PLEOF NPs, with their ability to release Paclitaxel for up to 4 weeks, are potentially useful for delivery of chemotherapeutic agents to tumors. The PLAF-NHS and PLGF-NHS NPs are also attractive for sustained delivery of growth and differentiation factors in tissue regeneration applications. When growth factors are administered as a solution or delivered in a collagen matrix, a large fraction of the protein is lost in the process of irrigating the wound. Furthermore, the use of injectable biomaterials and minimally invasive endoscopic techniques requires protein stabilization against denaturation during injection. Therefore, PLGF-NHS NPs are potentially useful for immobilization and sustained delivery of growth and differentiation factors in tissue engineering.

### CONCLUSIONS

Degradation of PLAF and PLGF NPs was followed by measuring their particle size and mass loss with time. For PLAF and PLGF NPs, particle size decreased significantly after 2 weeks and then increased slightly before degrading completely after 6 weeks for PLGF and 8 weeks for PLAF. Based on degradation results, a core-shell structure is proposed for the NPs in which the relatively more hydrophobic higher molecular weight fractions of PLAF/PLGF macromers undergo phase separation to form the core of the NPs followed by phase separation of the relatively less hydrophobic lower molecular weight fractions of PLAF/PLGF macromers and PLEOF to form the shell. The mass loss data indicated that the shell, with higher fraction of PLEOF than the core, constituted a large part of the volume of the NPs. The average PAN encapsulation efficiency in PLAF and PLGF NPs was 63±10%. The release profile of the encapsulated PAN was non-linear and consisted of a burst release in the first 24 h followed by a period of sustained release. The average BSA conjugation efficiency to PLAF-NHS and PLGF-NHS NPs was 61±20%. The release profile for the conjugated BSA for both PLAF-NHS and PLGF-NHS was linear up to complete degradation of the NPs. PLGF and PLAF NPs degraded in 15 and 28 days, respectively, while PLGF-NHS and PLAF-NHS NPs degraded in 25 and 38 days, which demonstrated that the release was dominated by matrix erosion. It should be noted that the hydrophilic end groups of PLGF and PLEOF macromers provided a driving force for diffusion of water in the NPs, resulting in the release of the small molecules from the NPs by diffusion and erosion.

In the interest of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims In addition, it should be understood that aspects of the various embodiments can be interchanged both in whole or in part. Furthermore, those of ordinary skill in the art will appreciate that the foregoing description is by way of example only, and is not intended to limit the disclosure so as further described in such appended claims.

What is claimed:

1. A method for forming a biodegradable composition that self-assembles into nanoparticles, the method comprising:
reacting N,N′-Disuccinimidyl carbonate with hydroxyl end-groups of poly(lactide-co-fumarate) to form a composition comprising succinimide-terminated poly(lactide-co-fumarate).

2. A method as in claim 1, further comprising combining the succinimide-terminated poly(lactide-co-fumarate) with poly(lactide-co-ethylene oxide-co-fumarate) in conditions that allow the composition to self-assemble into nanoparticles.

3. A method as in claim 2, wherein the succinimide-terminated poly(lactide-co-fumarate) is combined with poly(lactide-co-ethylene oxide-co-fumarate) in a solvent solution and dialysis is performed of the solution so that the composition self-assembles into nanoparticles.

4. A method as in claim 3, wherein the dialysis is performed against water.

6. A method as in claim 2, wherein the solvent comprises dimethylformamide, dimethylsulfoxide, or combinations thereof

7. A method as in claim 2, wherein the nanoparticles range from about 20 nm to about 1000 nm.

8. A method as in claim 2, wherein the nanoparticles range from about 50 nm to about 500 nm.

9. A method as in claim 2, further comprising conjugating a bioreactive protein to one or more nanoparticles.

10. A method as in claim 2, further comprising varying the size of the nanoparticles by adjusting the ratio of poly(lactide-co-fumarate) to poly(lactide-co-ethylene oxide-co-fumarate).

11. A method for forming a biodegradable composition that self-assembles into nanoparticles, the method comprising:
reacting N,N′-Disuccinimidyl carbonate with hydroxyl end-groups of poly(lactide-co-glycolide-co-fumarate) to form a composition comprising succinimide-terminated poly(lactide-co-glycolide-co-fumarate).

12. A method as in claim 10, further comprising combining the succinimide-terminated poly(lactide-co-glycolide-co-fumarate) with poly(lactide-co-ethylene oxide-co-fumarate) in conditions that allow the composition to self-assemble into nanoparticles.

13. A method as in claim 12, wherein the succinimide-terminated poly(lactide-co-glycolide-co-fumarate) is combined with poly(lactide-co-ethylene oxide-co-fumarate) in a solvent solution and dialysis is performed of the solution so that the composition self-assembles into nanoparticles.

14. A method as in claim 13, wherein the solvent comprises dimethylformamide, dimethylsulfoxide, or combinations thereof

15. A method as in claim 12, further comprising conjugating a bioreactive protein to one or more nanoparticles.

16. A method as in claim 12, further comprising varying the size of the nanoparticles by adjusting the ratio of poly(lactide-co-glycolide-co-fumarate) to poly(lactide-co-ethylene oxide-co-fumarate).

17. A method as in claim 12, wherein the nanoparticles range from about 20 nm to about 1000 nm.

18. A method as in claim 12, wherein the nanoparticles range from about 50 nm to about 500 nm.

19. A biodegradable composition that self-assembles into nanoparticles, the composition comprising succinimide-terminated poly(lactide-co-fumarate).

20. A biodegradable composition that self-assembles into nanoparticles, the composition comprising succinimide-terminated poly(lactide-co-glycolide-co-fumarate).
